(19) United States
(12) Patent Application Publication
(10) Pub. No.: US 2008/0260861 A1
(43) Pub. Date: Oct. 23, 2008

(54) Modulating Lymphatic Function

(75) Inventors: Jeroeng Hagendoorn, Boston, MA (US); Dai Fukumura, Newton, MA (US); Timothy P. Padera, Boston, MA (US); Rakesh K. Jain, Boston, MA (US)

Correspondence Address: FISH & RICHARDSON PC, P.O. BOX 1022, MINNEAPOLIS, MN, 55440-1022, US

(73) Assignee: THE GENERAL HOSPITAL CORPORATION, Boston, MA (US)

(21) Appl. No.: 11/547,884

(22) PCT Filed: Apr. 7, 2005
(86) PCT No.: PCT/US2005/011817

§ 371 Date: Jul. 8, 2008

Related U.S. Application Data
(60) Provisional application No. 60/560,078, filed on Apr. 7, 2004.

(51) Int. Cl. A61K 33/26; A61K 31/195; A61K 31/04; A61K 31/34; A61P 7/10; A61K 38/06

(52) U.S. Cl. 424/646; 514/565; 514/742; 514/470; 514/562; 514/18

(57) ABSTRACT

Methods and compositions for modulating lymphatic function, e.g., by altering NO levels, are disclosed.

### CROSS-REFERENCE TO RELATED APPLICATION

This application claims priority to U.S. Patent Application Ser. No. 60/560,078, filed on Apr. 7, 2004, the entire contents of which are incorporated by reference.

### GOVERNMENT FUNDING

This invention was made in part with Government support under Bioengineering Research Partnership Grant R24CA85140. The government has certain rights in the invention.

### BACKGROUND

Nitric oxide has been shown to relax smooth muscle cells (including vascular smooth muscle cells), inhibit vascular smooth muscle cell proliferation, protect endothelial cells from apoptosis, provide anti-thrombogenic and antioxidant effects, and promote wound healing.

### SUMMARY OF THE INVENTION

The invention is based, at least in part, on the inventors' discovery that lymphatic function, e.g., lymphatic flow, can be modulated in vivo by modulating levels of nitric oxide (NO) in the lymphatic system, e.g., in the collecting lymphatic vessels. More particularly, the inventors have found that decreasing NO, e.g., by inhibiting nitric oxide synthase (NOS), preferably eNOS, can decrease lymphatic flow and increasing NO, e.g., by administering an NO donor or substrate, can increase lymphatic flow. Accordingly, compositions and methods are described herein for modulating lymphatic function, e.g., lymphatic flow. The compositions and methods can be used, inter alia, to treat edema (e.g., by increasing NO), or to reduce lymphatic metastases (e.g., by decreasing NO).

In one aspect, the invention features a method of treating a subject, e.g., a subject in need of increased lymphatic flow, e.g., a subject identified as having, or at risk for, lymphedema, e.g., primary or secondary lymphedema. The method includes increasing nitric oxide (NO) or a response induced byNO, e.g., cGMP, in a lymphatic vessel (e.g., an initial lymphatic vessel or a collecting lymphatic vessel) of the subject. The subject is preferably a human, e.g., a human diagnosed with primary or secondary lymphedema.

In one embodiment, the method includes administering to the subject an agent that increases NO, e.g., an NO donor, e.g., L-arginine, sodium nitroprusside, nitroglycerin, glyceryl trinitrate, SIN-1, isosorbid mononitrate, isosorbid dinitrate, SNAP (S-nitroso-N-acetylpenicill), SNP (sodium nitroprusside), S-nitrosoglutathione, a NONOate (e.g., spenine NONOate or DEA-NONOate), L-homoarginine, N-hydroxy-L-arginine, a diazeniumdiolate (e.g., a polymer-based diazeniumdiolate). Also included are organic nitrates, O-nitrosylated compounds, S-nitrosylated compounds, NONOate compounds, inorganic nitroso compounds, sydnonimines (e.g., nits L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, inhibitors of the enzyme arginase (e.g., N-hydroxy-L-arginine and 2(S)-amino-6-boronohexanoic acid) and the substrates for nitric oxide synffiase, cytokines, adenosine, bradykinin, calreticulin, bisacodyl, and phenolphthalein. In many implementations, the agent is a non-proteinaceous agent. In some implementations, the agent is a proteinaceous agent that increases NO production, e.g., a growth factor such as a vascular endothelial growth factor (vascular endothelial growth factor-A, -C, or -D), angiopoietin-1, platelet derived growth factor; a molecule that affects the phophatidylinositol 3-kinase pathway to increase NO production, or a molecule that affects/increases cyclic GMP (cGMP) to increase NO production. In some implementations, the agent is an agent that increases cGMP, e.g., sildenafil or NO-sensitive guanylyl cyclase. In some implementations, the agent is an agent that increases Akt/Phosphokinase-C, e.g., VEGF, IGF, estrogen, or simvastatin. In some implementations, the agent is an agent that increases sphingosine 1-phosphate.

It is also possible to administer a combination of agents that increase NO, e.g., a non-proteinaceous compound and a proteinaceous compound (e.g., an NO donor and a growth factor), or two non-proteinaceous compound (e.g., two different NO donors)

In one embodiment, the subject has primary lymphedema. In another embodiment, the subject has secondary lymphedema, or is at risk for secondary lymphedema, e.g., the patient has undergone or will undergo a procedure that results in removal of, or damage to, the lymphatic system, e.g., the patient has undergone or will undergo surgery, radiation, infection or trauma that affects the lymphatic system

In some embodiment, the agent is administered in combination with one or more second treatments for lymphedema, e.g., manual lymphatic drainage, bandaging, pumps, compression garments, antibiotics, or diuretics.

In a preferred embodiment, the agent is administered via local administration to the affected tissue. For example, the agent is administered by topical application, transdermally, or subcutaneously in the area of the affected tissue.

In a preferred embodiment, the agent is administered in a lipid based formulation, e.g., a liposome or the agent is coupled to a lipophilic moiety. Such formulations can be administered, e.g., orally, e.g., to be taken up by the intestinal lymph, or topically.

In a preferred embodiment, the NO donor or substrate is coupled to a moiety, e.g., a macromolecule, that is preferentially taken up by lymphatic vessels relative to vascular vessels. For example, the agent can be coupled to a macromolecule that is preferably between about 10 and about 200 nm, e.g., between about 10 and about 50 nm, between about 50 and about 100 nm, between about 100 and about 150 nm, between about 150 and about 200 nm, or between about 50 and about 150 nm, preferably between about 50 and about 150 nm. The moiety can be, e.g., dextran (e.g., dextran having a mass of at least 100,000 Da; 500,000 Da; 1 million Da; 2 million Da) or a monoclonal antibody targeted to lymphatic vessels.

In certain cases, it may possible to deliver NO directly, e.g., deliver to a site where increased NO is required. The NO can be produced exogenously from the subject.

In some embodiments, the method includes evaluating the subject for one or more of: lymph node status, joint flexibility, skin fullness and/or tightness, and blood clots. The evaluation can be performed before, during, and/or after the administration of the agent. For example, the evaluation can be performed at least 1 day, 2 days, 4, 7, 14, 21, 30 or more days before and/or after the administration.

In a preferred embodiment, the administration of an agent can be initiated: when the subject begins to show signs of lymphedema; when lymphedema is diagnosed; at the time a treatment for lymphedema is begun or begins to exert its effects; before, during or following surgery, taa or radiation therapy, or generally, as is needed to maintain health.

The period over which the agent is administered (or the period over which clinically effective levels are maintained in the subject) can be long term, e.g., for six months or more or a year or more, or short term, e.g., for up to or less than a day, a week, two weeks, one month, three months, or six months.

In another aspect, the invention features a method of treating a subject, e.g., a subject in need of decreased lymphatic flow, e.g., a subject identified as having, or at risk for, a metastatic cancer, e.g., a lymphatic metastasis. The method includes decreasing nitric oxide (NO) in a lymphatic vessel of the subject. The subject is preferably a human, e.g., a human diagnosed with cancer, e.g., a subject diagnosed with a primary solid tumor. In some embodiments, the subject has undergone, or will undergo, surgery to remove a primary tumor.

In one embodiment, the method includes administering to the subject an agent that inhibits NO, e.g., a NOS inhibitor (preferably eNOS inhibitor), such as cavtratin, caveolin-1 scaffolding domain, NG—monomethyl-L-arginine (L-NMMA), N_{G}-nitro-L-arginine methyl ester (L-NAME), 2-ethyl-2-thiopseudourea (ETU), 2-methylisothiourea (SMT), 7-nitroindazole, aminoguanidine hemisulfate and diphenyleneiodonium (DPI). eNOS inhibitors are preferred. Also included are NO scavengers such as 2-phenyl-4,4,5,5-tetraethylimidazoline-1-oxyl-3-oxide (PTIO), 2-(4-carboxyphenyl)-4,4,5,5-tetraethylimidazoline-1-oxyl-3-oxide (Carboxy-PTIO) and N-methyl-D glucamine dithiocarbamate (MGD). Other exemplary agents that can inhibit eNOS include BN 80933, 7-nitroindazole, and DPI-chloride. The agent is typically a non-proteinaceous compound, but in certain cases may be proteinaceous. The agent can be less than 5000, 2000, 1000, or 500 Daltons in molecular weight.

In some embodiment, the agent is administered in combination with a second treatment for cancer or metastasis, e.g., one or more of: a chemotherapeutic agent, radiotherapy, an anti-angiogenic agent, an anti-lymphangiogenic agent.

In a preferred embodiment, the agent is administered via local administration to the affected tissue. For example, the agent is administered by topical application, transdermally, or subcutaneously in the area of the affected tissue, e.g., tissue at or near a site of a tumor.

In a preferred embodiment, the agent is administered in a lipid based formulation, e.g., a liposome or the agent is coupled to a lipophilic moiety. Such formulations can be administered, e.g., topically, subcutaneously, or orally, e.g., to be taken up by the intestinal lymph.

In a preferred embodiment, the NOS inhibitor or NO scavenger is coupled to a moiety, e.g., a macromolecule, that is preferentially taken up by lymphatic vessels relative to vascular vessels. For example, the agent can be coupled to macromolecule that is preferably between about 10 and about 200 nm, e.g., between about 10 and about 50 nm, between about 50 and about 100 nm, between about 100 and about 150 nm, between about 150 and about 200 nm, or between about 50 and about 150 nm, preferably between about 50 and about 150 nm. The moiety can be, e.g., dextran (e.g., dextran having a mass of at least 100,000 Da; 500,000 Da; 1 million Da; 2 million Da) or a monoclonal antibody targeted to lymphatic vessels.

In some embodiments, the method includes evaluating the subject for presence of neoplasia. The evaluation can be performed before, during, and/or after the administration of the agent. For example, the evaluation can be performed at least 1 day, 2 days, 4, 7, 14, 21, 30 or more days before and/or after the administration.

In a preferred embodiment, the administration of an agent can be initiated: when the subject begins to show signs of a tumor or cancer; when a tumor or cancer is diagnosed; at the time a treatment for a tumor or cancer is begun or begins to exert its effects; before, during or following surgery or therapy for a tumor or cancer, or generally, as is needed to maintain health.

The period over which the agent is administered (or the period over which clinically effective levels are maintained in the subject) can be long term, e.g., for six months or more or a year or more, or short term, e.g., for up to or less than a day, a week, two weeks, one month, three months, or six months. In another aspect, the invention features a method of decreasing lymphatic flow, e.g., in a subject who does not have a metastatic cancer (e.g., a lymphatic metastasis or a subject who is in need of reduced fat uptake. The method includes decreasing nitric oxide (NO) in a lymphatic vessel of the subject. The decrease in lymphatic flow can decrease lymphatic (and often systemic) uptake of certain cells or molecules, e.g. decrease fat uptake in the intestine, inflammatory cells/proteins at sites of inflammation (e.g., sites of intestinal or localized cutaneous infections), or decrease uptake of drugs targeted to specific sites and so forth.

The subject is preferably a human. For example, the subject has an inflammation or inflammatory disorder, e.g., an intestinal or localized cutaneous infection. In other examples, the subject is a subject in need of reduced inflammation or reduced fat uptake. In still another example, the subject is a subject who is receiving a drug therapy in which the drug is being targeted to specific sites.

In one embodiment, the method includes administering to the subject an agent that inhibits NO, e.g., a NOS inhibitor (preferably eNOS inhibitor), such as cavtratin, caveolin-1 scaffolding domain, N_{G}—monomethyl-L-arginine (L-NMMA), N_{G}-nitro-L-arginine methyl ester (L-NAME), 2-ethyl-2-thiopseudourea (ETU), 2-methylisothiourea (SMT), 7-nitroindazole, aminoguanidine hemisulfate and diphenyleneiodonium (DPI). eNOS inhibitors are preferred. Also included are NO scavengers such as 2-phenyl-4,4,5,5-tetraethylimidazoline-1-oxyl-3-oxide (PTIO), 2-(4-carboxyphenyl)-4,4,5,5-tetraethylimidazoline-1-oxyl-3-oxide (Carboxy-PTIO) and N-methyl-D-glucamine dithiocarbamate (MGD). Other exemplary agents that can inhibit eNOS include BN 80933, 7-nitroindazole, and DPI-chloride. The method can include other features described herein.

As used herein, a proteinaceous compound is one that includes at least three peptide bonds. Typically, a proteinaceous compound is polypeptide of greater than 20 amino acids. A non-proteinaceous compound is one that is not a proteinaceous compound.

This description also features the use of the compounds disclosed herein to provide the respective treatments suited for the compounds and to provide medicaments for such respective treatments.

### DESCRIPTION OF THE DRAWINGS

FIG. 1 is a schematic overview of mouse tail lymphangiography. Fluorescent dye is injected with constant pressure in the interstitium of the tail-tip for lymphatic uptake. Inset, intravital microscopy reveals a hexagonal network of initial lymphatics. RTD indicates region of intravital microscopy of lymphatic transport and residence time distribution analysis. Arrow indicates location of ligation of collecting lymphatic vessels.

FIG. 2 is a set of graphs showing the effects of NOS inhibition with L-NMMA and selective eNOS inhibition with Cavtratin on lymphatic function parameters. (A) Lymphatic fluid velocity (μm/s) is significantly lower in L-NMMA or Cavtratin treated animals than in controls treated with D-NMMA or AP, respectively. After ligation of the collecting lymphatics, this effect is eliminated. *p<0.005; **p<0.05; ***p<0.01. (B) Injection flow rate (nl/min) is significantly different between unligated and ligated controls. *p<0.05. (C) Mean lymphatic vessel diameter (slm) is not different between L-NMMA or Cavtratin treated animals and controls, but there is a significant difference between unligated and ligated controls. *p<0.001.

FIG. 3 shows eNOS is expressed in collecting lymphatics. Cross-sections through mouse tail prepared after ferritin lymphangiography. (A) Functioning initial lymphatic vessels (arrows) containing ferritin are highlighted green. A collecting lymphatic vessel (asterisk) can be identified as a larger ferritin containing structure adjacent to the tail vein (V). Scale bar denotes 100 μm. (B) eNOS is expression (arrows) is localized to the wall of collecting lymphatics containing ferritin (asterisk). The expression pattern resembles that of the tail vein (V). Scale bar denotes 15 μm.

FIG. 4 is a schematic representation of the effects of NO and ligation on the mousse tail lymphatic network. (A) The microlymphatic network consists of hexagonal initial lymphatics and two deep, collecting lymphatic vessels. Fluorescent tracer is injected with constant pressure in the interstitium of the distal end of the mouse tail. (B) In the physiological situation, the tracer is transported through the initial and collecting lymphatics. The latter have a muscular wall and intraluminal valves. (C) A constricted state of the collecting lymphatics during eNOS inhibition increases resistance and decreases fluid velocity in the lymphatic network. (D) Proximal ligation of the collecting lymphatics leaves the initial lymphatics as the only route for fluid flow. Loss of control of lymph fluid transport and decrease in total resistance, to which lymph vessel diameter is inversely proportional, leads to increased fluid velocity and injection flow rate.

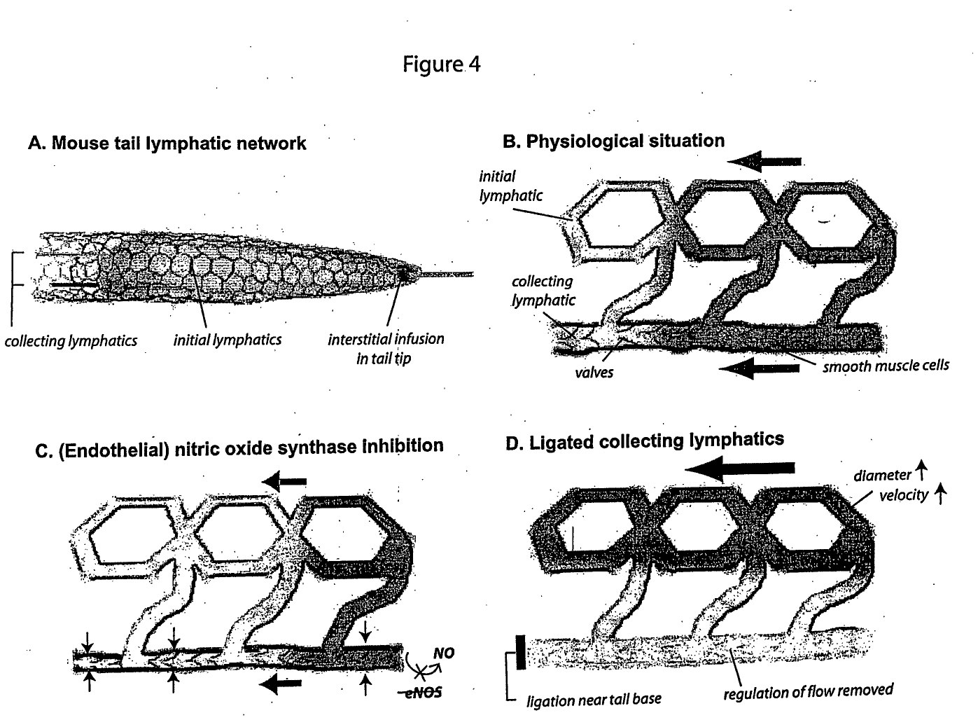

### DETAILED DESCRIPTION

The inventors have demonstrated a role for nitric oxide in regulating lymphatic function (e.g., lymphatic flow). Using an in-vivo model that permits intravital microscopy and microlymphangiography, it was found that NO synthase (NOS) inhibition decreased lymphatic fluid velocity in the initial lymphatics without an effect on their morphology. Using the same model, it was found that specific inhibition of endothelial NOS (eNOS) had a comparable effect. When the superficial, initial lymphatics are uncoupled from the deeper, collecting lymphatics by ligating the latter, it was found that lymphatic fluid velocity in NOS-inhibited mice became equal to that in control animals. Lymphatic fluid velocity was significantly increased after ligating the collecting lymphatics, and there was a concomitant increase in injection flow rate and mean lymphatic vessel diameter. Thus, eNOS affects function of the whole microlymphatic system and is regulated via the collecting lymphatics.

Accordingly, increasing NO, e.g., by administering an NO donor or substrate, provides a strategy to increase lymphatic flow, e.g., to treat a condition associated with decreased lymphatic flow, or a condition in which increasing lymphatic flow is desired, such as lymphedema. Decreasing NO, e.g., by administering a NOS inhibitor, provides a strategy to treat a medical condition associated with increased lymphatic flow, or a condition where decreased lymphatic flow is desirable, e.g., to reduce lymphatic metastases.

### The Lymphatic System

One of the principal functions of the lymphatic system is to collect and return interstitial fluid, including plasma protein to the blood, and thus help maintain fluid balance. In this function, first, interstitial fluid is taken up by blind-ended, capillary structures (˜60 μm in diameter) known as the initial lymphatics. These consist of adjacent lymphatic endothelial cells, which lack a continuous basement membrane and possess slight overlaps that act as primary valves. The initial lymphatics are dynamically coupled to the collagen fibers of the interstitium via anchoring filaments, so that increased interstitial volume and resultant radial tension on the lymphatics leads to increased convective interstitial-lymphatic fluid transport. Then, fluid is transported to larger lymphatic structures (100-150 μm in diameter) that have a smooth muscle layer and intraluminal valves, which divide the lymph vessels into functional units called lymphangions. From these collecting lymphatics, lymph fluid is transported, via lymph nodes and lymphatic trunks, to the thoracic duct and right lymphatic duct and, eventually, drained into the jugular and subclavian veins.

Determinants of lymph flow are extrinsic propulsive forces such as the lymph formation rate, respiration, and skeletal muscle movement, and the intrinsic contractility of the smooth muscle layer of the collecting lymphatics. Actual lymph flow rate depends on the interaction of these passive and active mechanisms. Although there is a positive pressure difference between the thoracic duct and dorsal foot lymphatics in humans in upright position, lymph flow is present during basal physiological conditions in caudocranial direction. It is speculated, therefore, that the contractile collecting lymphatics must act as a primary driving force for active lymph flow. A number of studies have confirmed systematic contractions of the collecting lymphatics in various ex vivo preparations. Moreover, oxygen tension is lower in mesenteric collecting lymphatics than in the surrounding interstitial fluid, corroborating in vivo energy consuming contractile processes of the lymphatic vessel wall. Thus, the transient contraction of each lymphangion forces fluid into the proximal lymphangion and, because one-way valves prevent backflow, this would result in net fluid flow towards the heart. The methods disclosed herein include methods that can be used to treat a subject in need of decreased lymph flow. Decreased lymph flow can be useful, e.g., in decreasing fat uptake in the intestine, uptake to the lymph of inflammatory cells or proteins at sites of inflammation, or in preventing lymphatic uptake of drugs targeted to specific sites. These methods can be used to treat subject who do not have cancer.

Many metastatic cancers spread through the lymphatic system. The methods disclosed herein can be useful in treating patients that have or are at risk for metastatic cancer, by decreasing lymphatic flow, e.g., generally or in the region of the tumor.

### Lymphedema

The methods disclosed herein can be useful for the treatment of patients that have, or are at risk for, lymphedema. Lymphedema is the accumulation of lymph in the interstitial spaces, principally in the subcutaneous fatty tissues, caused by a defect in the lymphatic system. It is marked by an abnormal collection of excess tissue proteins, edema, chronic inflammation, and fibrosis.

Lymphedema can be acquired after surgery or radiation therapy or caused at least in part by an infection, e.g., by a pathogen, e.g., an infection such as filariasis. Accordingly, methods for treating lymphadema (e.g., increasing NO) can be administered to a patient subject to surgery or radiation therapy, e.g., before, during, or after the surgery or therapy. The administration can be tailored, e.g., to localize increased NO, e.g., to a region affected by the surgery or therapy. Similarly, methods for treating lymphadema (e.g., increasing NO) can be administered to a patient subject to an infection or inflammation, e.g., an infection caused by filariasis.

Lymphedema can be categorized as primary or secondary. Primary, or congenital, lymphedema can occur locally or systemically and can have a genetic basis (e.g., a VEGFR3 mutation, or a FOXC2 mutation). Congenital forms of lymphedema usually manifest in the first few years of life, have a low global incidence, and can impose extreme morbidity on patients. Secondary, or acquired, lymphedema is generally caused by obstruction or interruption of the lymphatic system, which usually occurs at proximal limb segments (i.e., lymph nodes) due to infection, malignancy, or scar tissue. The pelvic and inguinal groups of nodes in the lower extremities and the axillary nodes of the upper extremities are the primary sites of obstruction.

Transient lymphedema is a temporary condition that lasts less than 6 months and is associated with pitting edema with tactile pressure and lack of brawny skin changes. The following factors may place the patient at risk for acute-onset, transient lymphedema: surgical drains with extravasation of protein into the surgical site; inflammation following injury, radiation, or infection leading to increased capillary permeability, immobility of the limb(s) that results in decreased external compression by the musculature; temporary absence of collateral lymphatics; proximal venous occlusion by thrombosis or phlebitis; and reversal of equilibrium at the capillary bed that results in accumulation of third-space fluid.

Chronic lymphedema can be difficult to reverse, due to the nature of its pathophysiology. A cycle is started, in which the deficient lymphatic system of the limb is incapable of compensating for the increased demand for fluid drainage. This condition may occur, e.g., subsequent to any of the following: tumor recurrence or progression in the nodal area; infection and/or injury of lymphatic vessels; immobility; radiation injury to lymphatic structures; surgery, unsuccessful management of early lymphedema; and venous obstruction due to thrombosis.

Early in the course of developing lymphedema, the patient can experience soft, pitting edema that may be easily improved by limb elevation, gentle exercise, and elastic support. Continual and progressive lymphostasis, however, causes dilation of the lymph vessels and backflow of fluid to the tissue beds. Collagen proteins accumulate, further increasing colloid osmotic tissue pressure, leading to enhanced fluid flow from the vascular capillaries into the interstitial space. The stasis of fluid and protein stimulates inflammation and macrophage activity as the body attempts to degrade the excess proteins. Fibrosis of the interstitial connective tissue by fibrinogen and fibroblasts causes the development of the brawny, stiff, nonpitting lymphedema that no longer responds to elevation, gentle exercise, or elastic compression garments. Chronic lymphedema gradually becomes nonpitting.

Lymphedematous tissues have lower oxygen content, a greater distance between lymph vessels due to fluid accumulation and swelling, impaired lymphatic clearance, and depressed macrophage function, rendering patients at increased risk of infection and cellulitis. Since there is no other route for tissue protein transport, treatment for patients with advanced lymphedema with chronic fibrosis is more difficult than when treated earlier. Additionally, once these tissues are stretched, edema recurs more readily.

Generalized lymphedema may also occur subsequent to hypoalbuminemia with low plasma oncotic pressure due to the following: inadequate oral nutrition (secondary to anorexia, nausea, vomiting, depression, chemotherapy); decreased intestinal absorption of protein or abnormal protein synthesis/anabolism; protein loss due to leakage of blood, ascites, effusions, or surgical drains; or contributing medical conditions leading to hypoalbuminemia (e.g., diabetes, kidney malfunction, hypertension, congestive heart failure, liver disease).

There appears to be an overall incidence of arm edema after breast cancer therapy of about 26%. Breast cancer patients (including ones treated by radiation or by surgery), particular those whose cancer is not a metastatic stage, and other subjects described herein can be administered a treatment for treating lymphedema, e.g., by increasing NO.

Water displacement measurement 15 cm above the epicondyle provides one exemplary and objective criterion with which to evaluate lymphedema; a displacement value of 200 mL included 96.4% of patients with subjective lymphedema. Some studies use 6 cm above the elbow; preferably, measurement of the upper extremities should be at consistent points along the arm, above and below the antecubital fossa, and across the hand or wrist.

Approximately 50% of patients with minimal edema report a feeling of heaviness or fullness of the extremity. Assessment of the patient with edema includes a history and physical examination. The history may include information regarding past surgeries, postoperative complications, prior radiation treatments, the time interval from radiation or surgery to the onset of symptoms, and intervening variables in the presence or severity of symptoms. The quality and behavior of the edema (fluctuation with position, progression over time) may be assessed. History of trauma or infection may be determined. In addition, information concerning current medications may be important. Edema is typically not detectable clinically until the interstitial volume reaches 30% above normal.

### Cancers and Metastatic Disorders

Examples of cancerous disorders include, but are not limited to, solid tumors, soft tissue tumors, and metastatic lesions thereof, including in particular those that may utilize the lymphatic system for metastasis. Examples of solid tumors include malignancies, e.g., sarcomas, adenocarcinomas, and cacinoma of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth Metastatic lesions of the aforementioned cancers, and particularly metastatic forms of these cancers, can also be treated or prevented using the methods and compositions described herein.

The method can be used to treat malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carm include those forming from tissue of the cervix, lung, prostate, breast, head and neck, colon and ovary. The term also includes carcinosarcoma, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is recognized by those skilled in the art and refers to malignant tumors of mesenchymal derivation.

### Nitric Oxide

Nitric oxide (NO) is synthesized by one of several isoforms of the NO synthase (NOS) family of enzymes, two of which are found in the vasculature, endothelial NOS (eNOS) and inducible NOS (iNOS). eNOS is synthesized by endothelial cells, while iNOS is synthesized by a variety of cell types, including vascular smooth muscle cells, fibroblasts, and (principally microvascular) endothelial cells (Balligand et al, Am J Physiol, 268:H1293-1303 (1995)). These enzymes produce NO as a result of the five-electron oxidation of L-arginine to L-citrulline.

Nitric oxide (NO) is a major regulator of microvascular function NO can also be generated by lymphatic endothelial cells (Shirasawa et al., Am J Physiol, 2000, 278:G551-G556). Lymphatic endothelial cells express nitric oxide synthase (NOS) in vivo and in vitro (Marchetti et al., Anat Rec., 1997, 248:490-497). Exogenous NO inhibits the pacemaking activity of lymphatic smooth muscle cells by activating protein kinases via the cyclic GMP pathway (Von der Weid, Br J. Pharmacol. 1998, 125:17-22). Applied NO was shown to resemble flow induced inhibition of contraction frequency of mesenteric lymphatics, while L-NMMA, a nitric oxide synthase inhibitor, could partially attenuate this effect (Gashev et al., J. Physiol. 2002, 540:1023-1037). Surprisingly, it is shown herein that increasing NO can increase lymphatic flow, e.g., in states of high lymph formation rate.

Agents That Increase NO

An ‘NO donor’ is a compound that releases nitric oxide or that acts as a substrate leading to the formation of nitric oxide.

A wide variety of nitric oxide donor compounds are available for the release and/or production of nitric oxide, including the following examples: organic nitrates (i.e., organic compounds having C—O—NO₂ groups), e.g., nitroglycerine; O-nitrosylated compounds (e.g., compounds, preferably organic, having —O—NO groups, these are also known as O-nitroso compounds or in some cases organic nitrites), e.g., isosorbide dinitrate, isosorbide mononitrate; S-nitrosylated compounds (e.g., compounds, preferably organic, having an —S—NO group, these are also known as S-nitroso compounds or S-nitrosothiols compounds, e.g., glutathione, S-nitrosylated derivatives of captopril, S-nitrosylated-proteins/peptides, S-nitrosylated oligosaccharides and polysaccharides, and so forth; NONOate compounds, e.g., substituted piperazines and diazeniumdiolates; inorganic nitroso compounds (e.g., inorganic compounds having —NO groups), e.g., sodium nitroprusside; sydnonimines; L-arginine (an enzyme substrate which leads to the formation of nitric oxide in vivo) and variants L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, and polypeptides comprising at least one of these amino acids.

Also included are compounds that upregulate NOS, e.g., eNOS, such as statins (e.g., simvastatin and mevastatin); agents that increase NO production, e.g., vascular endothelial growth factors (vascular endothelial growth factor-A, -C, or -D), angiopoietin-1, and platelet derived growth factor; molecules that affect the phophatidylinositol 3-kinase pathway; and molecules that affect/increase cyclic GMP. In some implementations, the agent is an agent that increases cGMP, e.g., sildenafil or NO-sensitive guanylyl cyclase. In some implementations, the agent is an agent that increases Akt/Phosphokinase-C, e.g., VEGF, IGF, estrogen, or simvastatin. In some implementations, the agent is an agent that increases sphingosine 1-phosphate.

Dosages of the nitric oxide donor compound(s) within the methods and compositions of the present invention will depend, for example, upon the size and age of the patient, the condition being treated/prevented, the nitric oxide donor compound(s) selected, the location of administration, the disposition of the nitric oxide donor compound (e.g., whether the nitric oxide donor compound is disposed on the surface of a medical article, within a matrix, within a solution/dispersion), and so forth. It is within the skill level of those of ordinary skill in the art to make such determinations.

Agents that Reduce NO

Suitable agents that reduce NO include NOS inhibitors such as N_{G}-monomethyl-L-arginine (L-NMMA), NO-nitro-L-arginine methyl ester (L-NAME),2-ethyl-2-thiopseudourea (ETU), 2-methylisothiourea (SMT), 7-nitroindazole, aminoguanidine hemisulfate and diphenyleneiodonium (DPI). eNOS inhibitors are preferred, e.g., cavtratin, caveolin-1 scaffolding domain. Also included are NO scavengers such as 2-phenyl-4,4,5,5-tetraethylimidazoline-1-oxyl-3-oxide (PTIO), 2-(4-carboxyphenyl)-4,4,5,5-tetraethylimidazoline-1-oxyl-3-oxide (Carboxy-PTIO) and N-methyl-D glucamine dithiocarbamate (MGD). Also known to inhibit eNOS are BN 80933, 7-nitroindazole, DPI-chloride. Other NOS inhibitors have been described in, e.g., Gapud et al., U.S. Pat. No. 5,981,511; Mjalli et al, U.S. Pat. No. 5,723,451; Hallinan et al., U.S. Pat. No. 6,143,790; Hansen et al., U.S. Pat. No. 6,071,906; Hansen et al., U.S. Pat. No. 6,043,261, all of which are herein incorporated by reference.

### Gene Therapy

A nucleic acid encoding an agent described herein, e.g., an NO-releasing agent, eNOS gene, or a nucleic acid that affects NO levels or eNOS or an antisense nucleic acid can be incorporated into a gene construct to be used as a part of a gene therapy protocol to deliver a nucleic acid encoding either an agonistic or antagonistic form of an agent described herein. Such expression constructs may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1, or recombinant bacterial or eukaryotic plasmids. Viral vectors transfect cells directly, plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or calcium phosphate precipitation carried out in vivo.

A preferred approach for in vivo introduction of nucleic acid into a cell is by use of a viral vector containing nucleic acid, e.g. a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Annentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhubits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flofte et al. (1993) J. Biol. Chem. 268:3781-3790).

In addition to viral transfer methods, such as those illustrated above, non-viral methods can also be employed to cause expression of a nucleic acid agent described herein (e.g., an eNOS encoding nucleic acid) in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding an agent described herein can be entrapped in liposomes bearing positive charges on their surface (e.g., lipofectins) and (optionally) which are tagged with antibodies against cell surface antigens of the target tissue (izuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system

### Administration

The agents described herein (e.g., NO donors or NOS inhibitors) may be formulated as pharmaceutical compositions administered via the parenteral route, including orally, topically, subcutaneously, intraperitoneally, intramuscularly, intranasally, and intravenously. More than one route of administration can be used simultaneously, e.g., topical administration in association with oral administration. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the NO modulating agents.

An agent described herein, an agent, e.g., an NO donor or NOS inhibitor, can be delivered by direct administration, e.g., injection (e.g., subcutaneously of intramuscularly). In one embodiment, the agent is delivered to an area of the body affected by lymphedema. The agent can be coupled to a second moiety, e.g., a delivery agent (e.g., an agent that targets the NO modulating agent to the lymphatic vessels, and/or an agent decreases the delivery of the agent to the blood circulatory system).

Local administration of the NO-modulating agents described herein is preferred and is described, e.g., in U.S. Pat. No. 6,706,274; U.S. Pat. No. 6,673,891; U.S. Pat. No. 6,656,217; U.S. Pat. No. 6,645,518.

In one embodiment, an S-nitrosylated β-cyclodextrin or an S-nitrosylated β-cyclodextrin complexed with S-nitroso-N-acetyl-D,L-penicillamine or S-nitroso-penicillamine or a nitrosylated polymer is used to increase NO.

### Kits

An NO-modulating agent, e.g., an agent described herein, can be provided in a kit. The kit includes (a) the agent, e.g., a composition that includes the agent, and (b) informational material. The informational material can be descriptive, instructional, marketing or other material that relates to the methods described herein and/or the use of the NO-modulating agent for the methods described herein. For example, the informational material relates to lymphedema or cancer.

In one embodiment, the informational material can include instructions to administer the NO-modulating agent in a suitable manner to perform the methods described herein, e.g., in a suitable dose, dosage form, or mode of administration (e.g., a dose, dosage form, or mode of administration described herein). Preferred doses, dosage forms, or modes of administration are topical, subcutaneous, and oral administration. In another embodiment, the informational material can include instructions to administer the NO-modulating agent to a suitable subject, e.g., a human, e.g., a human having, or at risk for, lymphedema or lymphatic metastasis.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about the NO-modulating agent and/or its use in the methods described herein. Of course, the informational material can also be provided in any combination of formats.

In addition to the NO-modulating agent, the composition of the kit can include other ingredients, such as a solvent or buffer, a stabilizer, a preservative, a fragrance or other cosmetic ingredient, and/or a second agent fo treating a condition or disorder described herein, e.g., the NO-modulating agent can be coated on a pressure bandage. Alternatively, the other ingredients can be included in the kit, but in different compositions or containers than the NO modulating agent. In such embodiments, the kit can include instructions for admixing the NO-modulating agent and the other ingredients, or for using the NO-modulating agent together with the other ingredients.

The NO-modulating agent can be provided in any form, e.g., liquid, dried or lyophilized form. It is preferred that the NO-modulating agent be substantially pure and/or sterile. When the NO-modulating agent is provided in a liquid solution, the liquid solution preferably is an aqueous solution, with a sterile aqueous solution being preferred. When the NO-modulating agent is provided as a dried form, reconstitution generally is by the addition of a suitable solvent. The solvent, e.g., sterile water or buffer, can optionally be provided in the kit.

The kit can include one or more containers for the composition containing the NO-modulating agent. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the NO-modulating agent. For example, the kit includes a plurality of syringes, ampules, foil packets, or blister packs, cream packs, each containing a single unit dose of the NO-modulating agent. The containers of the kits can be air tight and/or waterproof.

The kit optionally includes a device suitable for administration of the composition, e.g., a syringe, swab (e.g., a cotton swab or wooden swab), or any such delivery device. In a preferred embodiment, the device is a swab.

The data described in the following examples show, inter alia, that blocking NO through eNOS inhibition decreases lymphatic fluid velocity in the microlymphatic network and that this effect can be elirnated by functionally removing the collecting lymphatics. While not bound by theory, it is believed that collecting lymphatics respond to NO and provide outflow resistance to the initial lymphatics. The examples are not meant to limit the invention.

### EXAMPLES

### Example 1

### NOS Inhibition Decreases Initial Lymphatic Fluid Flow

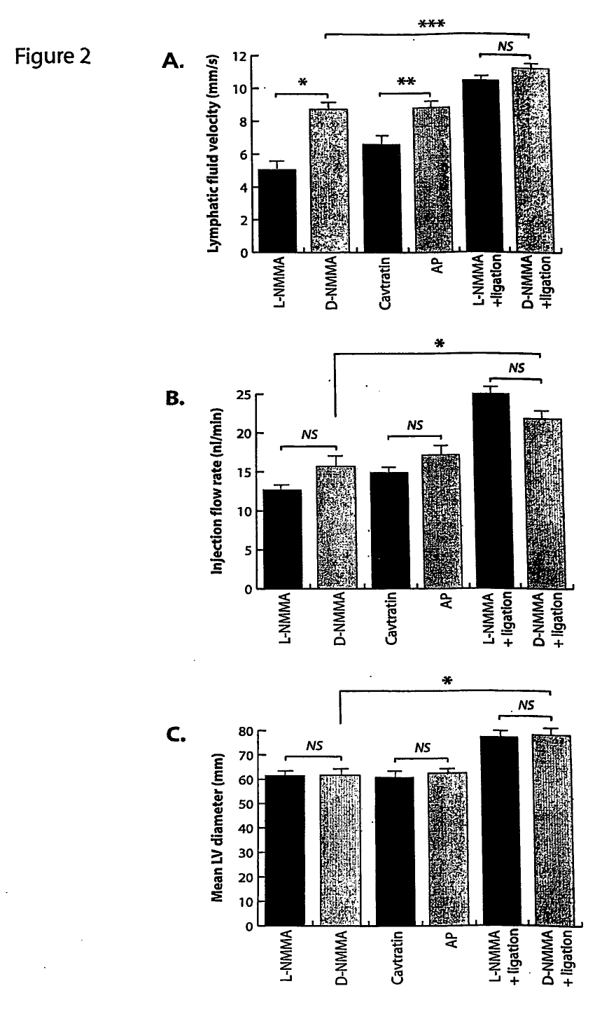

Lymphatic function measurements were performed in mice that had received 3 days of L-NMMA treatment for NOS inhibition, and it was found that overall lymphatic fluid velocity in the dermal lymphatic network was decreased by 42% compared to controls that had received D-NMMA (5.1±0.6 μm/s versus 8.7±0.4 μm/s, respectively; p<0.05) (FIG. 2A). The mean lymphatic fluid velocity in the control group receiving D-NMMA was comparable to that in mice without an infusion pump (8.7±0.4 μm/s versus 8.6±1.2 μm/s, respectively) as well as to that in humans. Injection rate of the fluorescent tracer into the interstitium was not significantly different between L-NMMA treated animals and controls (11.3±1.2 nl/min versus 15.7±1.5 nl/min, respectively; p=0.14) (FIG. 2B). In addition, there was no difference in mean lymphatic vessel diameter (61.5±0.7 μm versus 61.6±1.6 μm, respectively, NS) (FIG. 2C). To exclude a confounding effect of blood pressure at the time point studied, MAP via carotid artery cannulation was measured in a separate group of mice, and no difference was found between mice that had received L-NMMA and controls (71.7±1.4 mmHg versus 72.7±3.1 mmHg, respectively; NS). These data show that NOS inhibition decreases initial lymphatic fluid velocity without affecting mean lymphatic vessel diameter in the superficial network. The absence of a significant effect on injection rate should be interpreted with caution, since this is only an indirect indicator of lymphatic uptake. Although the collecting lymphatics are not directly functionally evaluated in this experiment, the regular connections between the initial and collecting lymphatics make it reasonable to assume that the time course of lymphatic filling in the deep, collecting lymphatics mirrors that in the superficial, initial network. Taken together, these data show that NOS inhibition decreases overall lymph flow.

### Example 2

### eNOS Inhibition Decreases Initial Lymphatic Fluid Flow

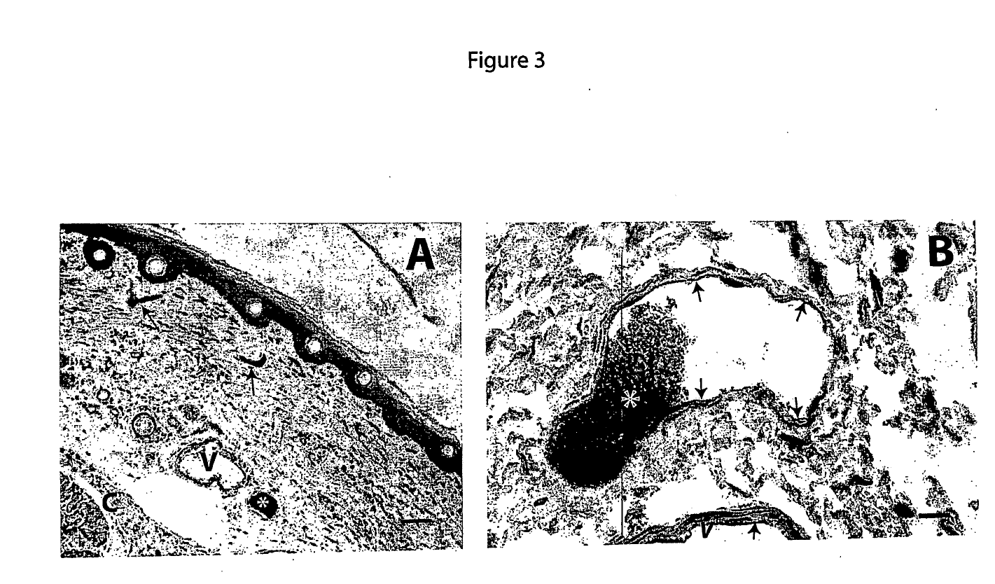

Immunohistochemistry was performed for eNOS, iNOS and neuronal NOS(nNOS) on tail sections, after ferritin lymphangiography to identify the lymphatic vessels. eNOS protein was localized to the walls of the collecting lymphatic vessels of the mouse tail (FIG. 3). There was no discernable staining of iNOS or NNOS in the lymphatics. Next, lymphatic function measurements were repeated in mice that had received the selective eNOS inhibitor Cavtratin for 3 days. Consistent with the L-NMMA treated animals, the overall lymphatic fluid velocity was decreased (6.6±0.3 μm/s versus 8.8±0.2 μm/s, respectively; p<0.05) (FIG. 2A). The injection rate of fluorescent tracer was not significantly different between Cavtratin treated animals and controls (14.9±0.7 μm/s versus 17.2±1.5 μm/s, respectively, NS) (FIG. 2B), nor was the mean lymphatic vessel diameter (60.7±2.3 μm/s versus 62.4±1.9 μm/s, respectively; NS) (FIG. 2C). These data show that the effects of NOS blockade on lymphatic function are mediated via eNOS. With the given dose of Cavtratin, previously shown to penetrate the interstitial space without systemic toxicity or an effect on blood pressure (Gratton et al., Cancer Cell 2003, 4:31-39; Bucci et al., Nat. Med. 2000; 12:1362-1367), lymphatic fluid velocity appeared less decreased compared to L-NMMA treated animals. Possibly, Cavtratin, at this dose, blocks a subfraction of eNOS proteins. Taken together, these data show that eNOS inhibition decreases lymphatic fluid flow.

### Example 3

### NOS Inhibition does not Affect Structure or Function of Uncoupled Initial Lymphatics

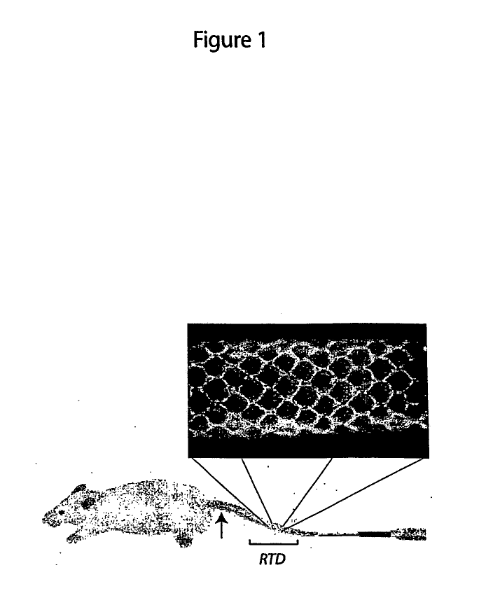

It was hypothesized that NOS inhibition affected lymphatic function via the collecting lymphatics. Therefore, the initial lymphatic network was uncoupled from the two deep, lateral collecting lymphatics by ligating the latter near the tail-base immediately before the experimental procedure (FIG. 1). After ligation, no significant difference in velocities was found between L-NMMA treated mice and controls (10.5±0.6 μm/s versus 11.2:±0.5 μm/s, respectively, NS) (FIG. 2A). In addition, there was no significant difference between the groups with respect to injection rate (25.0÷1.3 nl/min versus 21.8±1.9 nl/min, respectively, NS) (FIG. 2B) and mean lymphatic vessel diameter (77.2±2.1 μm versus 78.1±2.3 μm, respectively; NS) (FIG. 2C). After functionally removing the collecting lymphatics, the impairment of lymphatic fluid transport during NOS inhibition was eliminated. These data show that blocking NO through eNOS decreases lymphatic fluid velocity in the whole microlymphatic network, and this effect is mediated via the collecting, not the initial lymphatics.

### Example 4

### Initial Lymphatic Resistance is Decreased after Ligating the Collecting Lymphatics

To further examine the functional interaction between the initial and collecting lymphatic networks, lymphatic fluid velocity, injection flow rate, and mean lymphatic vessel diameter were compared between the non-ligated and the ligated control groups. In the ligated mice, the velocity was significantly higher than in non-ligated mice (11.2±0.5 μm/s versus 8.7∓0.4 μm/s, respectively, p<0.05) (FIG. 2A). In addition, the injection rate was increased in the ligated mice compared to non-ligated mice (21.8±1.9 nl/min versus 15.7±1.5 nl/min, respectively; p<0.05) (FIG. 2B), as was the mean lymphatic diameter (78.1±2.3 μm versus 61.6±1.5 μm, respectively, p<0.05) (FIG. 2C). The mean initial lymphatic vessel diameter is inversely proportional to initial lymphatic network resistance, which is decreased in the ligated group. Since the interstitial infusion pressure was kept constant, a lower resistance would result in a higher lymphatic fluid velocity and injection flow rate. These data strongly indicate that, in an intact microlymphatic network, the collecting lymphatics provide outflow resistance to the initial network and regulate overall lymph flow.

Materials and Methods

Animals

Studies were carried out in 7-10 week old female C57BL/6 and nude mice. Forty-eight mice were used for these experiments: sixteen for the inhibition experiments, fifteen for the ligation experiment, and seventeen additional controls. All procedures were carried out following the guidelines of the Institutional Animal Care and Use Committee of the Massachusetts General Hospital.

Experimental Design

Mice received a subcutaneous osmotic pump 3 days before the lymphatic function measurements for continuous infusion of L-NMMA or D-NMMA (controls) at 350 mg/kg daily, as described (Fukumura et al., (Abstract) Proceedings of AACR 2003, 44:471). For selective eNOS inhibition, mice received a daily intraperitoneal injection of Cavtratin at 2.5 mg/kg or the control peptide AP at 1.2 mg/kg, as described (Gratton et al., Cancer Cell 2003, 4:31-39), during 3 days before the lymphatic function measurements. The following groups were studied: group 1 (n=4), L-NMMA administration; group 2 (n=4), D-NMMA administration; group 3 (n=4), Cavtratin administration; group 4 (n=4), AP administration; group 5 (n=8), L-NMMA administration plus bilateral collecting lymph vessel ligation; group 6 (n=7), D-NMMA administration plus bilateral collecting lymph vessel ligation. Additional control groups consisted of: nude mice (n=3) to confirm that lymphatic fluid velocities were consistent with our previous data and C57BL/6 mice (n=4) without pump implantation.

Surgical Procedure

Mice in the experimental groups 5 and 6 underwent ligation of the deep collecting lymphatic vessels of the tail immediately before the microlymphangiography, to avoid development of edema (FIG. 1). Mice were anesthetized intramuscularly (90 mg/kg Ketamine and 9 mg/kg Xylazine) and placed on a heated surgical microscopy table. The translucent deep collecting lymphatic vessels were separated from the tail veins with microsurgical forceps through small, bilateral incisions in the axial direction, and ligated with a 10-O non-absorbable suture (Prolene, Ethicon, N.J., United States). The incision site was closed with surgical glue, taking care to avoid circumferential tension on the tail that could interfere with superficial lymphatic function.

Quantitative Lymph Flow Measurements Using Residence Time Distribution (RTD) Analysis

Fluorescence intensity measurements were carried out using RTD analysis as described previously (Swartz et al., Am J. Physiol. 1996, 270:H324-H329). Briefly, mice were anesthetized intramuscularly (90 mg/kg Ketamine and 9 mg/kg Xylazine) and placed on a small plate. 2.5% FITC-dextran (MW=2 million; Sigma, St Louis, Mo.) in PBS was infused into the interstitial tissue of the tail tip, with a constant pressure of 40 cm H₂O via a 30-gauge needle. Thus, changes in blood vessel permeability would not affect RTD measurements of initial lymphatic fluid velocity. The mouse was transferred to an epifluorescence microscopy setup as described previously (Leu et al., Am J. Physiol. 1994, 267:H1507-H1513). Eight adjacent fluorescent images of the tail, with a field dimension of 3.5 mm×2.5 mm, were obtained from distal to proximal, every ten minutes until saturation was reached in the most proximal region. The temporally consecutive fluorescent images were analyzed offline using NIH Image Analysis software. The average fluorescence intensity was determined for each image, and used to calculate the mean residence time for each region, the overall lymph fluid velocity in the tail lymphatic network, and the mean LV diameter.

Immunohistochemistry

Lymphatic vessels of the tail were histologically identified using ferritin lymphangiography (type I ferritin, Mᵣ 480,000; Sigma Chemical Co.) as described (Leu et al., Cancer Res 2000, 60:4324-4327). Distribution of the NOS isoforms on lymphatic vessel walls was examined immunohistochemically using monoclonal antibodies against eNOS, iNOS, and nNOS (Transduction Laboratory, Inc).

Mean Arterial Blood Pressure

8 week old, female C57BL/6 mice were weighed and anesthetized (90 mg/kg Ketamine and 9 mg/kg Xylazine). Mean arterial pressure (MAP) was measured by cannulating the exposed left carotid artery with a PE-10 intravascular polyethylene catheter, connected to a pressure transducer (Gould Inc, Valley View, Ohio). MAP was measured for 15 minutes, after 3 days of L-NMMA administration (n=5) and compared with PBS controls (n=3).

Statistics

Results are presented as mean ±SE. Student's t-test (equal variances not assumed) was used to evaluate statistical significance (defined as p<0.05).

### EQUIVALENTS

Those skilled in the art will recognize, or be able to ascertain using no more than routine experimentation, many equivalents to the specific embodiments of the invention described herein. Such equivalents are intended to be encompassed by the following claims.

All references (inclusive of patents and patent applications) disclosed herein are incorporated by reference in their entirety.

### REFERENCES

1. Alderton et al., Nitric oxide synthases: structure, function and inhibition. Biochem J. 2001; 357:593-615.
2. Aukland and Reed, Interstitial-lymphatic mechanisms in the control of extracellular fluid volume. Physiol Rev. 1993; 73:1-78. Schmid-Schönbein, Microlymphatics and lymph flow. Physiol Rev. 1990; 70:987-1028.
3. Benoit et al., Characterization of intact mesenteric lymphatic pump and its responsiveness to acute edemagenic stress. Am J. Physiol. 1989; 257:H2059-H2069.
4. Boucher et al., Ultrastructural comparative study on lymphatic capillaries of the subendocardium, myocardium, and subepicardium of the heart left ventricle. Microvasc Res. 1985; 29:305-319.
5. Bucci et al., In vivo delivery of the caveolin-1 scaffolding domain inhibits nitric oxide synthesis and reduces inflammation Nat. Med. 2000; 12:1362-1367.
6. Fischer et al., Flow velocity in single lymphatic capillaries in human skin. Am J Physiol. 1996; 270:H358-H363.
7. Fukumura et al., Predominant role of endothelial nitric oxide synthase in vascular endothelial growth factor-induced angiogenesis and permeability. Proc Natl Acad Sci USA. 2001; 98:2604-2609.
8. Fukumura et al., Host endothelial nitric oxide synthase mediates angiogenesis in murine melanomas. (Abstract) Proceedings of AACR 2003; 44:471.
9. Gashev et al., Inhibition of the active lymph pump by flow in rat mesenteric lymphatics and thoracic duct J. Physiol. 2002; 540:1023-1037.
10. Gratton et al., Selective inhibition of tumor microvascular permeability by cavtratin blocks tumor progression in mice. Cancer Cell 2003; 4:31-39.
11. Hangai-Hoger et al., Microlymphatic and tissue oxygen tension in the rat mesentery. Am J. Physiol. 2003; published online November 20.
12. Huang et al., Hypertension in mice lacking the gene for endothelial nitric oxide synthase. Nature 1995; 377:239-242.
13. Ignarro et al., Nitric oxide as a signaling molecule in the vascular system: an overview. J Cardiovasc Pharmacol. 1999; 34:879-886.
14. Kashiwagi et al., Nonendothelial source of nitric oxide in arterioles but not in venules. Circ Res. 2002; 91:e55-64.
15. Leu et al., Flow velocity in the superficial lymphatic network of the mouse tail. Am J. Physiol. 1994; 267:H1507-H1513.
16. Leu et al., Absence of functional lymphatics within a murine sarcoma: a molecular and functional evaluation. Cancer Res 2000; 60:4324-4327.
17. Marchetti et al., Endothelin and nitric oxide synthase in lymphatic endothelial cells: immunolocalization in vivo and in vitro. Anat Rec. 1997; 248:490-497.
18. Muthuchamy et al., Molecular and functional analyses of the contractile apparatus in lymphatic muscle. FASEB J. 2003; 17:920-922.
19. Olszewski and Engeset, Intrinsic contractility of prenodal lymph vessels and lymph flow in human leg. Am J. Physiol. 1980; 239:H775-H783.
20. Padera et al., Lymphatic metastasis in the absence of functional intratumor lymphatics. Science 2002; 296:1883-1886
21. Pullinger and Florey, Some observations on the structure and functions of the lymphatics: their behaviour in local edema. Br J Exp Pathol. 1935; 16:49-69.
22. Shirsawa et al., Physiological roles of endogenous nitric oxide in lymphatic pump activity of rat mesentery in vivo. Am J. Physiol. 2000; 278:G551-G556.
23. Slavin et al., Return of lymphatic function after flap transfer for acute lymphedema Ann Surg. 1999; 229:421-427.
24. Swartz et al., Transport in lymphatic capillaries. I. Macroscopic measurements using residence time distribution theory. Am J. Physiol. 1996; 270:H324H329.
25. Trzewik et al., Evidence for a second valve system in lymphatics: endothelial microvalves. FASEB J. 2003; 15:1711-1717.
26. Von der Weid, ATP-sensitive K+ channels in smooth muscle cells of guinea-pig mesenteric lymphatics: role in nitric oxide and beta-adrenoreceptor agonist-induced hyperpolarizations. Br J. Pharmacol. 1998; 125:17-22.

We claim:

1. A method of treating a subject that has or is at risk for lymphedema, the method comprising increasing nitric oxide (NO) in a lymphatic vessel of the subject.

2. The method of claim 1, wherein increasing NO comprises administering to the subject an NO donor.

3. The method of claim 2, wherein the NO donor is selected from the group consisting of: L-arginine, sodium nitroprusside, nitroglycerin, glyceryl trinitrate, SIN-1, isosorbid mononitrate, isosorbid dinitrate, S-nitroso-N-acetylpenicillamine (SNAP), sodium nitroprusside (SNP), S-nitrosoglutathione, NONOates, and diazeniumdiolates.

4. The method of claim 2, wherein the NO donor is administered via local administration to an affected tissue.

5. The method of claim 4, wherein the NO donor is administered by topical application, transdermally, or subcutaneously in the area of an affected tissue.

6. The method of claim 5, wherein the NO donor is selected from the group consisting of: L-arginine, sodium nitroprusside, nitroglycerin, glyceryl trinitrate, SIN-1, isosorbid mononitrate, isosorbid dinitrate, S-nitroso-N-acetylpenicillamine (SNAP), sodium nitroprusside (SNP), S-nitrosoglutathione, NONOates, and diazeniumdiolates.

7. The method of claim 5 wherein the NO donor is an O-nitrosylated compound or an S-nitrosylated compound.

8. The method of claim 6, wherein the NO donor is L-arginine.

9. The method of claim 2, wherein the NO donor is formulated in a lipid based delivery system.

10. The method of claim 2, wherein the NO donor is coupled to a moiety of sufficient size to be preferentially taken up by lymphatic vessels relative to vascular vessels.

11. The method of claim 10, wherein the moiety is between about 10 and about 200 nm.

12. The method of claim 1, wherein the subject has primary lymphedema.

13. The method of claim 1, wherein the subject has secondary lymphedema.

14. The method of claim 1, wherein the subject is a human.

15. The method of claim 14, further comprising, before, during, or after, the increasing, performing surgery or radiation therapy on the subject.

16. The method of claim 14, wherein the subject has an infection.

17. A method of treating a human subject that has lymphedema, the method comprising increasing nitric oxide (NO) in a lymphatic vessel of the subject by locally administering to the subject an NO donor in an area affected by lymphedema.

18. The method of claim 17, wherein the NO donor is selected from the group consisting of: L-arginine, sodium nitroprusside, nitroglycerin, glyceryl trinitrate, SIN-1, isosorbid mononitrate, isosorbid dinitrate, S-nitroso-N-acetylpenicillamine (SNAP), sodium nitroprusside (SNP), S-nitrosoglutathione, NONOates, and diazeniumdiolates.

19. The method of claim 18, wherein the subject has lymphedema caused by surgery, radiation therapy, or an infection.

20. A method of increasing lymphatic flow in a subject, the method comprising increasing nitric oxide (NO) in a lymphatic vessel of the subject by administering to the subject an NO donor.

21. A method of treating a subject in need of decreased lymphatic flow, the method comprising: identifying a subject in need of decreased lymphatic flow, and decreasing nitric oxide (NO) in a lymphatic vessel of the subject.

22. The method of claim 21, wherein decreasing NO comprises administering to the subject at least one agent selected from a NOS inhibitor and an NO scavenger.

23. The method of claim 22, wherein the agent is selected from the group consisting of: N_{G}—monomethyl-L-arginine (L-NMMA), N_{G}-nitro-L-arginine methyl ester (L-NAME), 2-ethyl-2-thiopseudourea (ETU), 2-methylisothiourea (SMT), 7-nitroindazole, aminoguanidine hemisulfate and diphenyleneiodonium (DPI), 2-phenyl-4,4,5,5-tetraethylimidazoline-1-oxyl-3-oxide SPIRO), 2-(4-carboxyphenyl)-4,4,5,5-tetraethylimidazoline-1-oxyl-3-oxide (Carboxy-PTIO) and N-methyl-D-glucamine dithiocarbamate (MGD), BN 80933, 7-nitroindazole, and DPI-chloride.

24. The method of claim 22, wherein the agent is administered via local administration to a tissue in need of decreased lymphatic flow.

25. The method of claim 22, wherein the agent is administered by topical application, transdermally, or subcutaneously in an area of the subjects body in need of decreased lymphatic flow.

26. The method of claim 22, wherein the agent is formulated in a lipid based delivery system.

27. The method of claim 22, wherein the agent is coupled to a moiety of sufficient size to be preferentially taken up by lymphatic vessels relative to vascular vessels.

28. The method of claim 27, wherein the moiety is between about 10 and about 200 m.
